(19) United States
(12) Patent Application Publication
(10) Pub. No.: US 2012/0121707 A1
(43) Pub. Date: May 17, 2012

(54) Tolbutamide Particle And Preparing Method Thereof And Method Of Reducing A Blood Glucose

(75) Inventors: Yan-Ping Chen, Taipei (TW); Pai-Ching Lin, Taipei (TW)

(73) Assignee: National Taiwan University, Taipei (TW)

(21) Appl. No.: 12/944,637

(22) Filed: Nov. 11, 2010

(51) Int. Cl. A61K 9/14; C07C 273/02; A61P 3/10; A61K 31/17

(52) U.S. Cl. 424/489; 514/592; 564/42

(57) ABSTRACT

A method for preparing a tolbutamide particle is provided. The method comprises steps of mixing a bulk drug of tolbutamide with a supercritical fluid to form a supercritical mixture; and expanding the supercritical mixture to obtain the tolbutamide particle.

### FIELD OF THE INVENTION

The present invention relates to the tolbutamide particles, the preparing method thereof and the method of reducing the blood glucose by using the tolbutamide particle, especially to the tolbutamide micro-particles, the method of preparing the tolbutamide micro-particles by adopting the technique of the rapid expansion of supercritical solution (RESS), and the method of reducing the blood glucose by using the tolbutamide micro-particles.

### BACKGROUND OF THE INVENTION

The tolbutamide is one of sulfonylurea antidiabetic agents, and was used to treat Typhoid Fever in the past. Since it was found that the tolbutamide can reduce the blood glucose of the patients of Typhoid Fever, the tolbutamide was developed as the oral drug for reducing the blood glucose, and commercialized with the trademark Orinase® in US market.

The tolbutamide drugs currently in the market have the minimum particle size about 90 micron. The molecular structure of the tolbutamide is shown below. The molecular weight of the tolbutamide is 270.35 g/mol, its melting point is in a range of 128 to 130° C., and its pKa value is 5.3. Therefore, the tolbutamide is weak acid, almost insoluble in the water, but soluble in ethanol and chloroform. In addition, the tolbutamide is a compound with multiple crystalline types, including four different crystalline types, i.e. form I, form IL form III and form IV.

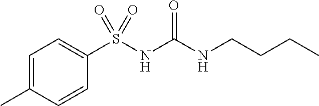

It is known that the tolbutamide has the functional mechanism of increasing the concentration of 3′-5′-cyclic adenosine monophosphate (C-AMP) to stimulate the pancreatic β cells to release insulin and then to reduce the blood glucose. Thus, the tolbutamide can be used to treat the patients with insulin-independent diabetes, i.e. type II diabetes, since the cause of the type II diabetes results from conditions that the β cells can not release enough insulin. On the other side, the time period of the pharmaceutical effect of the tolbutamide molecule is relatively short, the tolbutamide molecule can be metabolized to inactive metabolite quickly, and accordingly the tolbutamide molecule can be used to treat the patients with the kidney disease.

However, the particle sizes of the tolbutamide drugs currently available in the market are too large, and accordingly the tolbutamide drugs can not be dissolute quickly enough. Furthermore, the particle size distribution of the tolbutamide drug currently available in the market is too broad for the pharmaceutical applications.

For overcoming the mentioned drawbacks existing in the conventional techniques, the tolbutamide micro-particles with very small particle sizes, the pharmaceutical applications of the tolbutamide micro-particles and the novel methods for preparing the tolbutamide micro-particles are provided in the present invention with the great advantages of the fast dissolution rate, excellent medical effect, outstanding stability and low production cost.

### SUMMARY OF THE INVENTION

The present invention provides the pharmaceutical application of the tolbutamide particles and the method for preparing the tolbutamide particles with the minimized particle size.

In accordance with one aspect of the present invention, a method for preparing a tolbutamide particle is provided. The method comprises steps of a) mixing a bulk drug of tolbutamide with a supercritical fluid to form a supercritical mixture; and b) expanding the supercritical mixture to obtain the tolbutamide particle.

In accordance with another aspect of the present invention, a method of reducing a blood glucose of an animal, comprising a step of administering to the animal in need thereof a tolbutamide particle having one of average particle sizes smaller than and equal to 80 micron.

In accordance with a further aspect of the present invention, a tolbutamide particle having one of average particle sizes smaller than and equal to 80 micron is provided.

The above objects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1A is the schematic diagram showing the distribution and the cumulative distribution of the particle sizes for the bulk drug of tolbutamide;

FIG. 1B is the schematic diagram showing the distribution and the cumulative distribution of the particle sizes for the tolbutamide micro-particles obtained by using the method of the rapid expansion of supercritical solution (RESS) in the fourth embodiment;

FIG. 1C is the schematic diagram showing the distribution and the cumulative distribution of the particle sizes for the tolbutamide micro-particles obtained by using the method of RESS with solid cosolvent (RESS-SC) in the eighth embodiment;

FIG. 1D is the schematic comparison diagram showing the cumulative distributions of the particle sizes for the tolbutamide micro-particles obtained by RESS method (fourth embodiment) and by RESS-SC method (eighth embodiment);

FIG. 2A is the diagram showing the scanning electron microscopy for the bulk drug of tolbutamide;

FIG. 2B is the diagram showing the scanning electron microscopy for the tolbutamide micro-particles obtained by using the RESS method in the fourth embodiment;

FIG. 2C is the diagram showing the scanning electron microscopy for the tolbutamide micro-particles obtained by using the RESS-SC method in the eighth embodiment;

FIG. 3 is the schematic diagram showing the expansion processes of the RESS and RESS-SC methods for the comparison;

FIG. 4 shows the Fourier Transform Infrared (FTIR) spectra for (A) the bulk drug of tolbutamide, (B) menthol, (C) the tolbutamide micro-particles made by the RESS-SC method and (D) the tolbutamide micro-particles made by the RESS-SC method and purified;

FIG. 5A is the diagram of the differential scanning calorimetry (DSC) for the bulk drug of tolbutamide;

FIG. 5B is the DSC diagram for the tolbutamide micro-particles in the fifth embodiment;

FIG. 5C is the DSC diagram for the tolbutamide micro-particles in the eighth embodiment;

FIG. 6A is the diagram of the X-ray diffraction (XRD) for the bulk drug of tolbutamide;

FIG. 6B is the XRD diagram for the tolbutamide micro-particles in the fifth embodiment;

FIG. 6C is the XRD diagram for the tolbutamide micro-particles in the eighth embodiment;

FIG. 7 shows the Fourier Transform Infrared (FTIR) spectra for (A) the bulk drug of tolbutamide, (B) the tolbutamide micro-particles in the fifth embodiment and (C) the tolbutamide micro-particles in the eighth embodiment;

FIG. 8A is the visible-ultraviolet absorbance spectra for the tolbutamide;

FIG. 8B is the diagram showing the standard curve of the visible-ultraviolet absorbance of the tolbutamide vs. the concentration of the tolbutamide; and

FIG. 9 is the diagram showing the comparative curves of the dissolution rates for the bulk drug of tolbutamide, the tolbutamide micro-particles made by the RESS method in the fourth embodiment and the tolbutamide micro-particles made by the RESS-SC method in the eighth embodiment.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

The present invention is related to the method of preparing the tolbutamide particles. The method includes the following steps: a) mixing a bulk drug of the tolbutamide with a supercritical fluid to form a supercritical mixture, and b) quickly expanding the supercritical mixture to precipitate the tolbutamide micro-particles. Any appropriate supercritical fluids can be selectively adopted in the methods of the present invention. The supercritical fluid plays the role of the solvent. Since the carbon dioxide is non-toxic, colorless, odorless, incombustible and non-corrosive, the harm to the environment is low. Furthermore, it is easy to reach the critical point, i.e. critical pressure 73.8 bar and critical temperature 31.1° C., of the carbon dioxide, and accordingly the carbon dioxide is selected as the supercritical fluid in the preferred embodiments of the present invention.

According to the method of the present invention, in the step a) of the present embodiment, the dependency of solubility of the solute, i.e. tolbutamide, on the density of the solvent of the supercritical fluid is utilized, and preferably the supercritical mixture of the supercritical fluid and the tolbutamide is in a saturation state so that the soluble amount of the tolbutamide in the supercritical fluid can reach a maximum.

In order to promote the efficiency of manufacturing the tolbutamide particle, the step a) can further include mixing a solid cosolvent, e.g. menthol. Generally, it is found that the homogeneous phase formed by mixing the solid cosolvent with the supercritical mixture can further increase the solubility of the solute, i.e. tolbutamide, and accordingly the yield rate of the whole production can be increased. The usage amount of the solid cosolvent can be adjusted, depending on the adopted conditions of the method, e.g. the variety of the supercritical fluid. For instance of the solid cosolvent, the menthol, and the supercritical fluid, carbon dioxide, the usage amount of the solid cosolvent is ranged from 10% to 50% in weight, preferably from 20% to 40% in weight, relative to the amount of the tolbutamide in weight.

The step a) of the present embodiment is performed in the supercritical condition to obtain a supercritical mixture, which contains the tolbutamide, the supercritical fluid and the solid cosolvent. For example, in case that the carbon dioxide is adopted as the supercritical fluid, the step a) is performed under the pressure of 130 to 250 bar and the temperature of 305 to 328 K, preferably under the pressure of 140 to 220 bar and the temperature of 310 to 323 K. If the higher pressure or temperature than the above is adopted, the production cost will be increased, and the risk of operating the production apparatus will be increased, too. On the other hand, if the lower pressure or temperature than the above is adopted, the yield rate of the production can not be satisfied.

The step b) in the present invention is performed by quickly expanding the supercritical mixture obtained in the step a). The “quickly expanding” in the present invention means that the volume of the supercritical mixture is quickly (or instantaneously) expanded, so that solubility of the solute, tolbutamide, in the supercritical fluid is quickly dropped. In one embodiment, the quick expansion of the supercritical mixture is performed by quickly reducing the pressure, e.g. from the supercritical pressure dropped to the normal pressure, of the supercritical mixture, so that the state of the supercritical mixture is then changed from the saturation state under the supercritical conditions to the extremely over-saturation state under the normal pressure. Accordingly, the tolbutamide originally solved in the supercritical fluid is instantaneously precipitated, and the tolbutamide micro-particles can be obtained. It is believed that the tolbutamide in the step b) undergoes the following processes: reaching the over-saturation state, forming the crystal nucleus and crystal growing, i.e. crystallization processes, so as to obtain the tolbutamide micro-particles with the desired particle sizes (or particle diameters).

The instantaneous phase change of the supercritical fluid is utilized in the step b) of the present invention to control the solubility of the solute so as to obtain the tolbutamide micro-particles. Therefore, in addition to utilizing the pressure reduction to transform the state of the supercritical fluid from the supercritical state before the expansion to the gaseous state after the expansion, the method of reducing the temperature can be simultaneously adopted to accelerate the above transforming process. According to the one embodiment of the present invention, in the step b), the temperature before the expansion is controlled in a range of 380 to 405 K, and that after the expansion is controlled in a range of 275 to 295 K. Preferably, the temperature before the expansion is controlled in a range of 385 to 395 K, and that after the expansion is controlled in a range of 280 to 290 K.

As the above mention, the cosolvent can be added in the step a) of the present invention to increase the solubility of the tolbutamide in the supercritical fluid. In such conditions, the collision phenomenon between the tolbutamide molecules during the quick expansion in the step b) can be further diminished due to the existence of the cosolvent so that the tolbutamide micro-particles with smaller particle sizes (or particle diameters) can be advantageously obtained.

Besides, generally the temperature before the expansion is controlled to be higher than the melting point of the tolbutamide in order to prevent the precipitation of the tolbutamide before the step b) to block the apparatus due to the temperature difference, since the steps a) and b) of the present invention are performed in the different temperature ranges.

When the solid cosolvent is used, the method of the present invention can further include the purification of the tolbutamide micro-particles obtained in the step b) to remove the residue cosolvent so as to further promote the quality of the tolbutamide micro-particles. Generally, the tolbutamide micro-particles can be treated under the vacuum condition to evaporate and to remove the residue solvent.

The present invention is related to the tolbutamide micro-particles, which average particle size is smaller than or equal to 80 micron (micrometer, μm), preferably smaller than 50 micron, further preferably smaller than 25 micron, particularly preferably smaller than 10 micron and most preferably smaller than 3 micron. That is to say, the tolbutamide micro-particles obtained by using the method of the present invention can have the average particle size smaller than those (generally about 90 micron) of the bulk drug of tolbutamide currently in the market, and can greatly enhance the bio-utilization of the tolbutamide drug.

It is found that the crystal form of the tolbutamide particle can be turned to form II when the solid cosolvent is used in the step a). Compared to the bulk drug of tolbutamide with the crystal form I, the tolbutamide particle with the crystal form II has fast dissolution rate, accordingly can be quickly absorbed in the digestive tracts in the human body, and provides the better pharmaceutical effects.

The present invention is also related to the pharmaceutical application of the tolbutamide micro-particles on the medicine. The medicine can be used to reduce the blood glucose, and specially can be used to treat the type II diabetes. The tolbutamide micro-particles in the present invention provide the great advantages in the pharmaceutical applications due to their excellent dissolution rate,

### Embodiments 1 to 4

In the embodiments 1 to 4, the usage amount of the bulk drug of tolbutamide is in a range of 2 to 5 gram, and the other operation conditions, e.g. the pressures and the temperature of mixing, the temperatures before and after the expansion and the particle sizes of the tolbutamide micro-particles, are listed in Table 1 below.

TABLE 1

| | | | Temp. | Temp. | | | Average | |
|---|---|---|---|---|---|---|---|---|
| | Mixing | Mixing | before | after | Injection | Nozzle | particle | Standard |
| | pressure | temp. | expansion | expansion | distance | diameter | size | deviation |
| Embodiments | (bar) | (K) | (K) | (K) | (cm) | (μm) | (μm) | (μm) |
| Bulk drug of | | | | | | | 89.4 | 41.2 |
| tolbutamide | | | | | | | | |
| 1 | 150 | 308 | 393 | 283 | 2.5 | 50 | * | * |
| 2 | 150 | 318 | 393 | 283 | 2.5 | 50 | * | * |
| 3 | 200 | 308 | 393 | 283 | 2.5 | 50 | 9.2 | 4.9 |
| 4 | 200 | 318 | 393 | 283 | 2.5 | 50 | 8.5 | 5.1 |

* unanalyzed

The standard deviation (SD) is defined as

$$\sqrt{\frac{1}{N - 1} {\sum }_{j = 1}^{N} {( {X}_{j} - \overline{X} )}^{2}}$$

### Embodiments 5 to 8

In the embodiments 5 to 8, the usage amount of the bulk drug of tolbutamide is in a range of 2 to 5 gram, and the other operation conditions, e.g. the pressures and the temperature of mixing, the temperatures before and after the expansion and the particle sizes of the tolbutamide micro-particles, are listed in Table 2 below.

TABLE 2

| | | | Temp. | Temp. | | | Average | |
|---|---|---|---|---|---|---|---|---|
| | Mixing | Mixing | before | after | Injection | Nozzle | particle | Standard |
| | pressure | temp. | expansion | expansion | distance | diameter | diameter | deviation |
| Embodiments | (bar) | (K) | (K) | (K) | (cm) | (μm) | (μm) | (μm) |
| 5 | 150 | 308 | 393 | 283 | 2.5 | 50 | 2.7 | 1.4 |
| 6 | 150 | 318 | 393 | 283 | 2.5 | 50 | 2.9 | 1.3 |
| 7 | 200 | 308 | 393 | 283 | 2.5 | 50 | 2.4 | 1.2 |
| 8 | 200 | 318 | 393 | 283 | 2.5 | 50 | 2.1 | 0.9 |

In the present invention, the bulk drug of tolbutamide is treated by using the RESS method and the RESS-SC method, and the obtained tolbutamide micro-particles are compared with the bulk drug of tolbutamide. In the embodiments 1 to 8, the temperature before the expansion is 393 K, and the temperature after the expansion is 283 K. Two sets of mixing pressures are adopted (150 bar for the embodiments 1, 2, 5 and 6; 200 bar for the embodiments 3, 4, 7 and 8) and two sets of mixing temperatures are adopted (308 K for the embodiments 1, 3, 5 and 7; 318 K for the embodiments 2, 4, 6 and 8) for the steps of mixing the supercritical carbon dioxide and the bulk drug of tolbutamide.

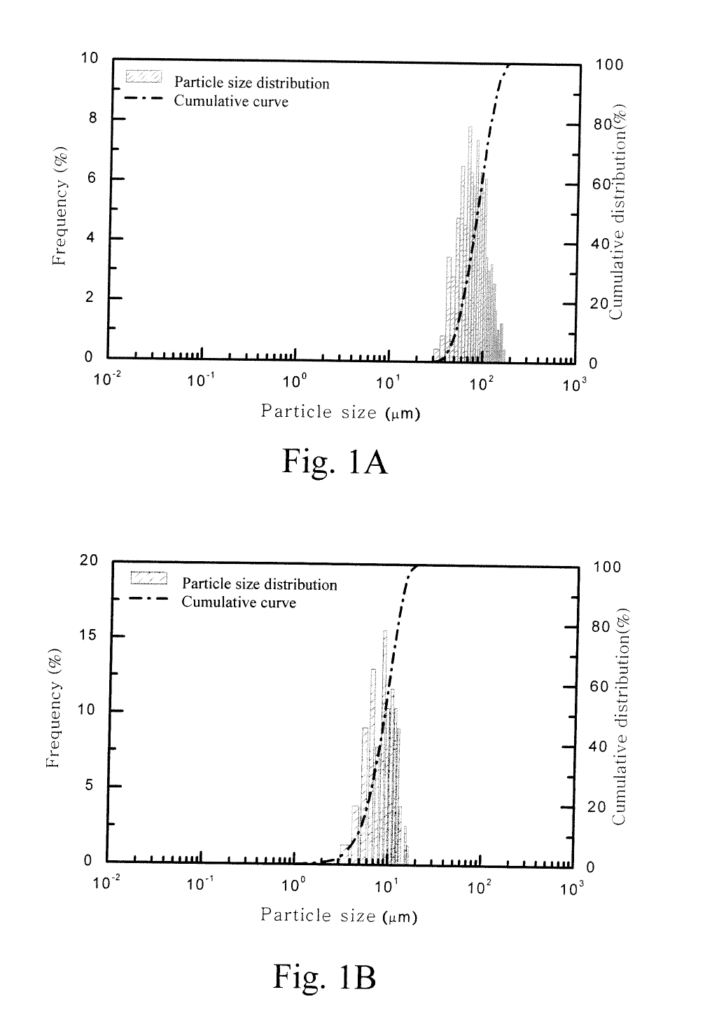

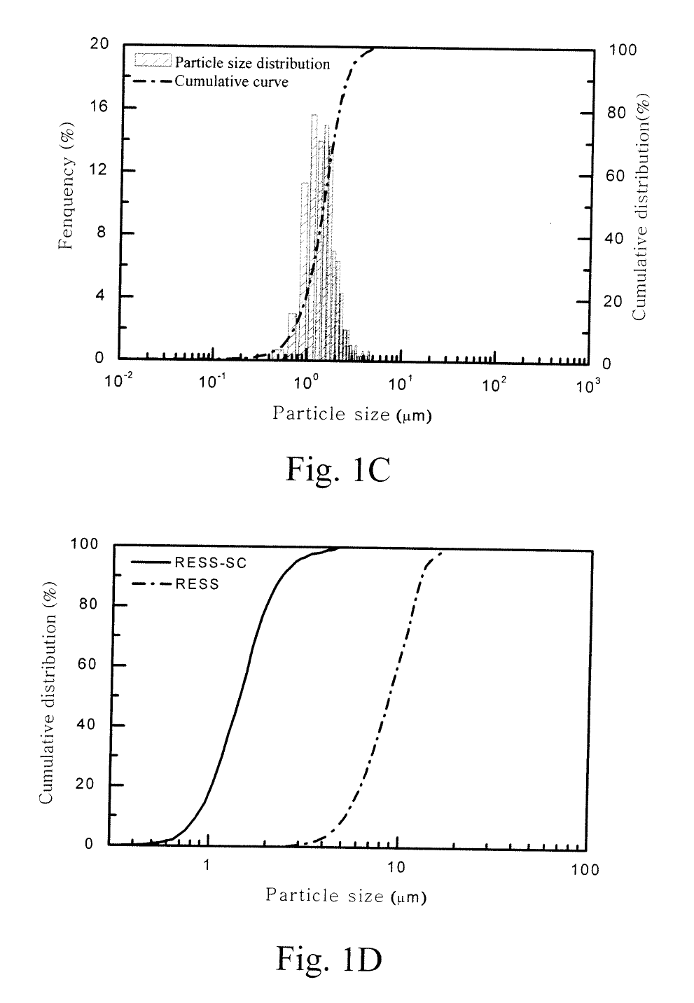

FIG. 1A is the schematic diagram showing the distribution and the cumulative distribution of the particle sizes for the bulk drug of tolbutamide, wherein the average particle size is 89.4 micron. FIG. 1B is the schematic diagram showing the distribution and the cumulative distribution of the particle sizes for the tolbutamide micro-particles obtained by using the method of the rapid expansion of supercritical solution (RESS) in the fourth embodiment. FIG. 1C is the schematic diagram showing the distribution and the cumulative distribution of the particle sizes for the tolbutamide micro-particles obtained by using the method of RESS with solid cosolvent (RESS-SC) in the eighth embodiment. The results in FIGS. 1B and 1C can fully prove the particle minimization effect of the present invention. FIG. 1D provides the comparison for the cumulative curves of the particle sizes in FIGS. 1B and 1C. It can be clearly seen in FIG. 1D, the average particle size of the tolbutamide micro-particles made by the RESS-SC method is 2.1 micron; while he average particle size of the tolbutamide micro-particles made by the RESS method is 8.5 micron, and both the average particle sizes are much smaller than the average particle size, 89.4 micron, of the bulk drug of tolbutamide in the current market.

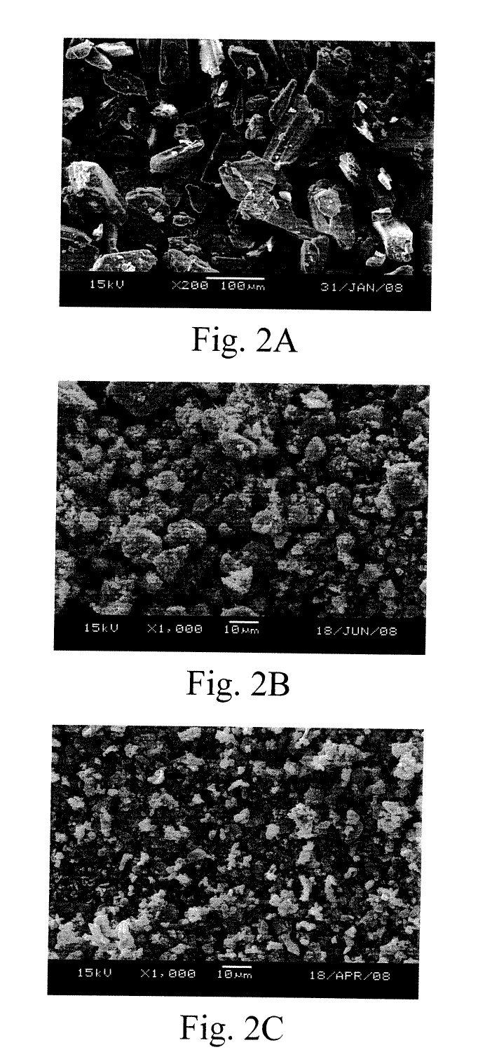

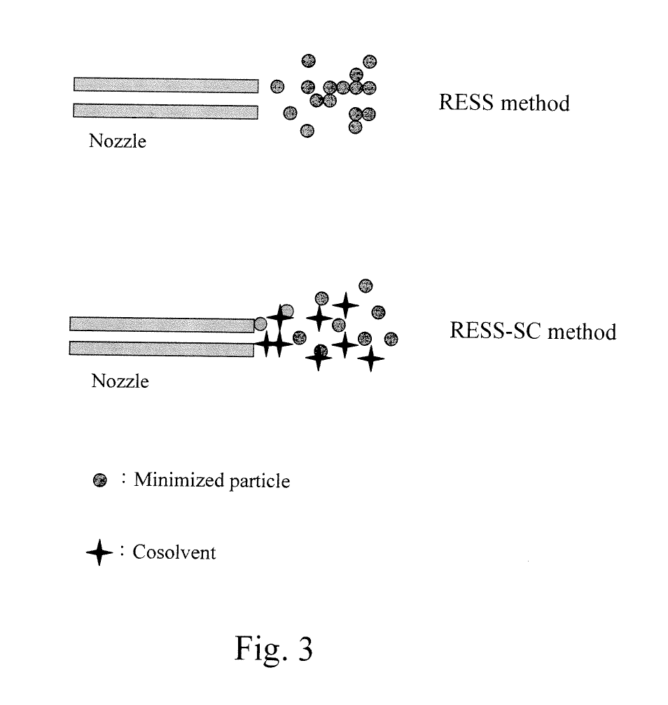

FIGS. 2A-2C are the diagrams of the scanning electron microscopy (SEM). It can be seen form FIG. 2A that the bulk drug of tolbutamide has a long strip shape. FIG. 2B is the diagram showing the scanning electron microscopy for the tolbutamide micro-particles obtained by using the RESS method in the fourth embodiment, wherein the shape of the tolbutamide micro-particles is about ellipsoidal and blocky. FIG. 2C is the diagram showing the scanning electron microscopy for the tolbutamide micro-particles obtained by using the RESS-SC method in the eighth embodiment, wherein the tolbutamide micro-particles are loosely flake micro-particles with much smaller particle sizes than those of the bulk drug of tolbutamide. It can be proved from these results that by introducing the solid cosolvent, the solubility of tolbutamide in the supercritical carbon dioxide can be increased, and the phenomenon of the collisions between the tolbutamide molecules to amass together can be effectively diminished. As illustrated in FIG. 3, the tolbutamide micro-particles with smaller particle sizes and uniform distribution of the particle sizes can be obtained due to the existence of the solid cosolvent.

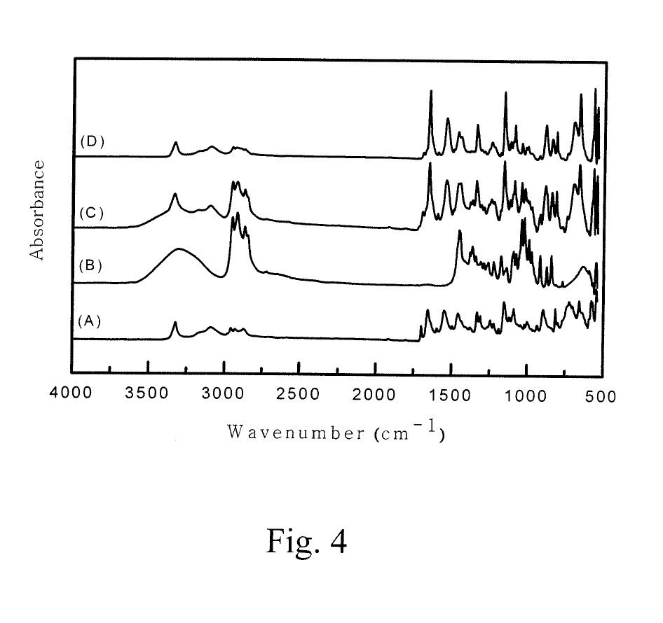

FIG. 4 is the Fourier Transform Infrared (FTIR) spectra for (A) the bulk drug of tolbutamide, (B) menthol, (C) the tolbutamide micro-particles made by the RESS-SC method and (D) the tolbutamide micro-particles made by the RESS-SC method and purified. The (C) of FIG. 4 shows the signal peaks of the functional group of menthol in 2759 and 2998 cm⁻¹, which can be identified by those in (B) for the menthol. From the spectrum of (D) in FIG. 4, it can be understood that the tolbutamide micro-particles after the purification contain no menthol. That is, the residue menthol in the tolbutamide micro-particles has been removed through the purification process.

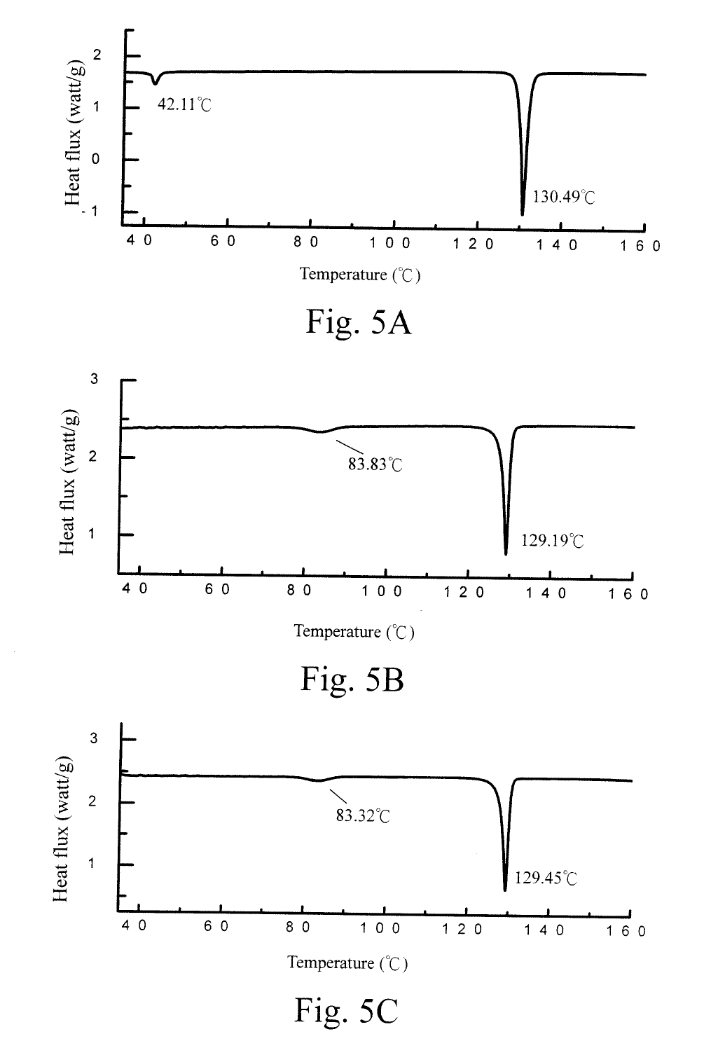

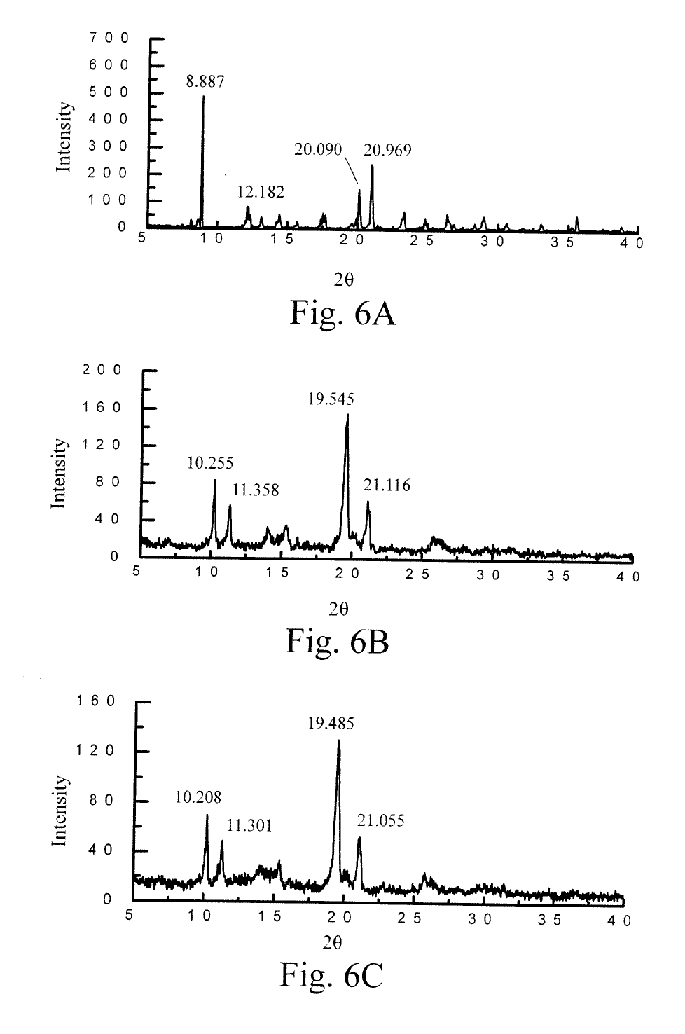

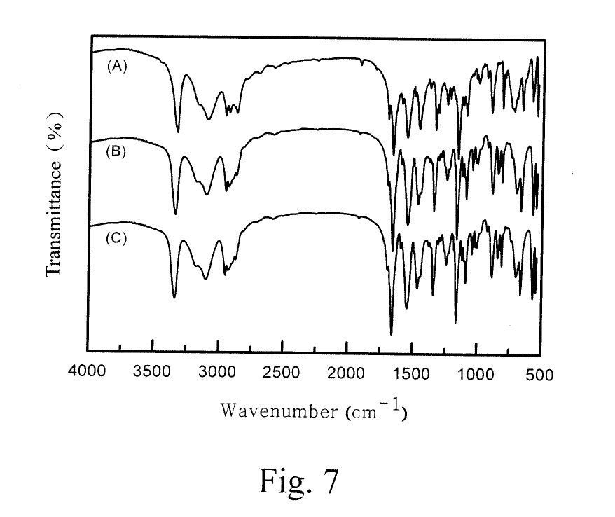

FIG. 5A is the diagram of the differential scanning calorimetry (DSC) for the bulk drug of tolbutamide. FIG. 5B is the DSC diagram for the tolbutamide micro-particles in the fifth embodiment. FIG. 5C is the DSC diagram for the tolbutamide micro-particles in the eighth embodiment. FIGS. 5A-5C show the transforms of the crystal forms for the tolbutamides. Compared with the FIG. 5A, there is a respective new absorption peak appearing around 83° C. in both FIGS. 5B and 5C for the tolbutamide micro-particles made by the RESS-SC method. In addition, it can be understood from the X-ray diffraction (XRD) spectra in FIGS. 7A-7C that the crystal forms of the tolbutamide micro-particles made by the RESS-SC method in the fifth and eighth embodiments, shown in FIGS. 7B and 7C, are much different from that of the bulk drug of tolbutamide shown in FIG. 7A.

FIG. 7 shows the Fourier Transform Infrared (FTIR) spectra for (A) the bulk drug of tolbutamide, (B) the tolbutamide micro-particles in the fifth embodiment and (C) the tolbutamide micro-particles in the eighth embodiment. It can be observed that the signal intensity in the range of 546 to 1134 cm⁻¹ for (B) and (C) in FIG. 7 are different from that for (A) in FIG. 7. Therefore, it can be understood that the tolbutamide micro-particles made by the RESS-SC method have the crystal form II transformed from the original form I for the bulk drug of tolbutamide based on the above XRD and FTIR analyses.

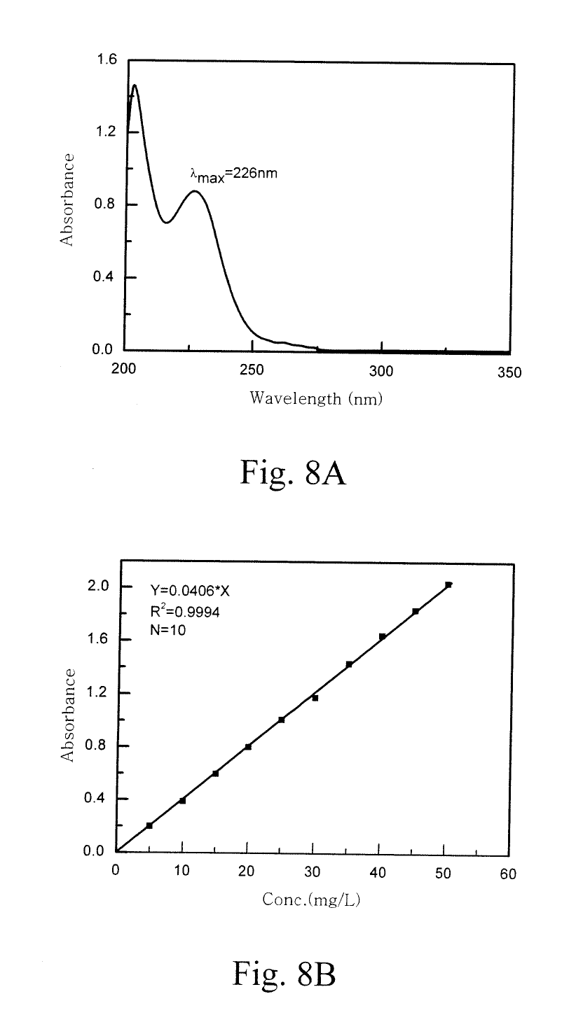

In the dissolution analyses for the bulk drug of tolbutamide and the tolbutamide micro-particles made by the method of the present invention, the operational and analytical conditions are set by conforming to United State Pharmacopeia (USP) 2008. FIG. 8A is the visible-ultraviolet absorbance spectra for the tolbutamide. FIG. 8B is the diagram showing the standard curve of the visible-ultraviolet absorbance of the tolbutamide vs. the concentration of the tolbutamide. The characteristic absorption peak for the tolbutamide is located at 226 nm as shown in FIG. 8A.

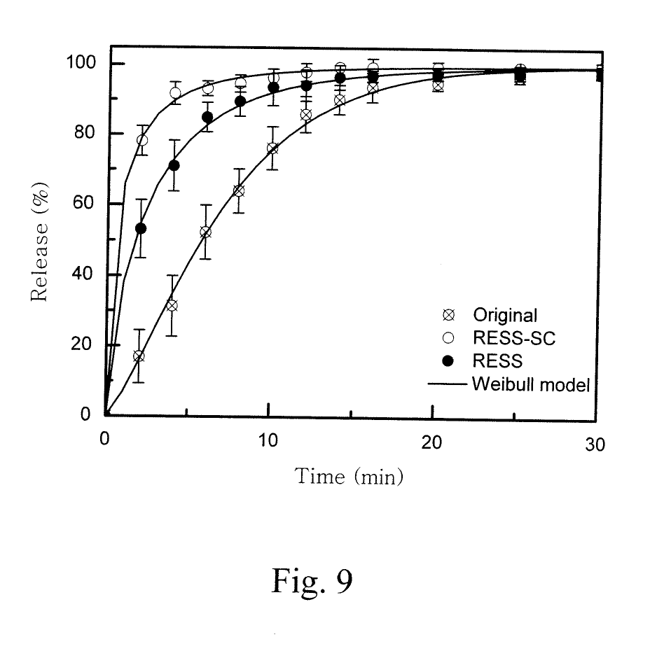

In the analyses of the dissolution, the amount of 40 mg for each of the original bulk drug of tolbutamide, the tolbutamide micro-particles from the fourth embodiment and the tolbutamide micro-particles from fifth embodiment are used. As shown in FIG. 9, the tolbutamide micro-particles made by the RESS-SC method in the eighth embodiment have the fastest dissolution rate, the tolbutamide micro-particles made by the RESS method in the fourth embodiment have the second fastest dissolution rate, and both the tolbutamide micro-particles in the fourth and eight embodiments have much higher dissolution rate than that of the original bulk drug of tolbutamide. In addition to the above analyses of the particle sizes and dissolution rates, the crystal form II for the tolbutamide micro-particles made by the RESS-SC method may contribute to the fast dissolution rate.

It can known from the above analyses that the tolbutamide micro-particles made by the methods of the present invention have excellent dissolution rate much higher than that of the bulk drug of tolbutamide currently available in the market. Therefore, the tolbutamide micro-particles made by the methods of the present invention can be applied to the field of the medicines, e.g. drugs for reducing blood glucose, treating the type II diabetes, etc., with the outstanding advantages.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A method for preparing a tolbutamide particle, comprising steps of:
a) mixing a bulk drug of tolbutamide with a supercritical fluid to form a supercritical mixture; and
b) expanding the supercritical mixture to obtain the tolbutamide particle.

2. A method of claim 1, wherein the supercritical fluid comprises a carbon dioxide, and the supercritical mixture is in a saturation state.

3. A method of claim 1, wherein the step a) further comprises mixing a solid cosolvent.

4. A method of claim 3, wherein the cosolvent comprises a menthol.

5. A method of claim 3, wherein the cosolvent has an amount of 10 to 50 weight percentage relative to that of the bulk drug of tolbutamide.

6. A method of claim 5, wherein the amount of the cosolvent is in a range of 20 to 40 weight percentage relative to that of the bulk drug of tolbutamide.

7. A method of claim 3, further comprising a step of purifying the tolbutamide particle obtained in the step b) under a vacuum condition to remove a residue cosolvent.

8. A method of claim 1, wherein the step a) is performed under a pressure of 130 to 250 bar and a temperature of 305 to 328 K.

9. A method of claim 8, wherein the pressure is in a range of 140 to 220 bar and the temperature is in a range of 310 to 323 K.

10. A method of claim 1, wherein the expansion in the step b) is performed by reducing a pressure.

11. A method of claim 10, wherein the step b) has a temperature before the expansion in a range of 380 to 405 K and a temperature after the expansion in a range of 275 to 295 K.

12. A method of claim 11, wherein the temperature before the expansion is in a range of 385 to 395 K, and the temperature after the expansion is in a range of 285 to 290 K.

13. A method of claim 10, performed by using a rapid expansion of supercritical solution (RESS) method.

14. A method of reducing a blood glucose of an animal, comprising a step of:
administering to the animal in need thereof a tolbutamide particle having one of average particle sizes smaller than and equal to 80 micron.

15. A method of claim 14, wherein the animal in need thereof has type II diabetes.

16. A tolbutamide particle having one of average particle sizes smaller than and equal to 80 micron.

17. A tolbutamide particle of claim 16, wherein the average particle size is smaller than 25 micron.

18. A tolbutamide particle of claim 16, wherein the average particle size is smaller than 10 micron.

19. A tolbutamide particle of claim 16, wherein the average particle size is smaller than 3 micron.

20. A tolbutamide particle of claim 16, having a crystal type of form II.
